(19) United States
(12) Patent Application Publication
(10) Pub. No.: US 2025/0099198 A1
(43) Pub. Date: Mar. 27, 2025

(54) FLEXIBLE ENDOSCOPE STORAGE HOOK, CABINET AND SYSTEM

(71) Applicant: OLYMPUS Winter & Ibe GmbH, Hamburg (DE)

(72) Inventor: Stefan SCHROEDER, Hamburg (DE)

(73) Assignee: OLYMPUS Winter & Ibe GmbH, Hamburg (DE)

(21) Appl. No.: 18/891,326

(22) Filed: Sep. 20, 2024

Related U.S. Application Data
(60) Provisional application No. 63/539,842, filed on Sep. 22, 2023.

(51) Int. Cl. A61B 50/22; A61B 50/10; A61B 90/98; A61M 25/00; G16H 40/20

(52) U.S. Cl. CPC A61B 50/22; A61B 90/98; G16H 40/20; A61B 2050/105; A61M 25/002

(57) ABSTRACT

A storage hook for storing a flexible endoscope. The storage hook including: a receptacle for storing the flexible endoscope, an endoscope presence sensor configured to detect a placement or removal of the flexible endoscope in the receptacle, and a controller including a processor comprising hardware. The processor being configured to receive an input signal from the sensor and to output an output signal based on the input signal from the sensor.

### CROSS-REFERENCE TO RELATED APPLICATION

The present application is based upon and claims the benefit of priority from U.S. Provisional Application No. 63/539,842 filed on Sep. 22, 2023, the entire contents of which is incorporated herein by reference.

### BACKGROUND

### Field

The present disclosure relates to a flexible endoscope storage hook.

### Prior Art

The reprocessing of flexible endoscopes requires an adequate drying and storage after high-level disinfection. Customers can choose from a variety of product offerings such as drying and/or storage cabinets. Some customers refrain from buying such fully equipped drying and/or storage cabinets and instead construct their own storage cabinets e.g. from standard furniture or within a dedicated room. These self-built solutions can be compliant with latest standards and are cost-effective.

However, these self-built solutions lack several features compared to drying and storage cabinets. Especially, the individual track and trace of a single endoscope is not possible or only with additional manual steps. Control parameters such as storage time, operator identification or documentation of environmental parameters such as temperature and humidity, which are customary in commercially available drying and/or storage cabinets, cannot be processed automatically. Therefore, operating a reprocessing workflow with self-built storage solutions entails substantial disadvantages relative to documentation and process control. This will result in negative impact on overall infection prevention and control.

### SUMMARY

An object is to provide a flexible endoscope storage hook, cabinet and/or system for avoiding the above-described problems even in self-built storage solutions.

Such object can be solved by a flexible endoscope storage hook comprising a receptacle for a flexible endoscope, an endoscope presence sensor configured to detect a placement or removal of a flexible endoscope in the receptacle, and a controller.

In embodiments, the controller can be configured and equipped for process control and/or documentation. Such process control and/or documentation may comprise one or more of track and trace of one or more individual flexible endoscopes, control parameters such as storage time, operator identification or documentation of environmental parameters such as temperature and humidity.

Such a hook having its own sensor and controller can also be installed in commercially available, simple storage cabinets and take over the aforementioned tasks for process control and/or documentation.

The storage hook can be equipped with storage and process control that is commonly implemented into commercially available drying and storage cabinets. In order to implement this functionality, in embodiments the controller can comprise a CPU, a display and a wireless connection unit. Wireless connectivity such as Wi-Fi or Bluetooth may be used to integrate one or more flexible endoscope storage hooks into a system providing centralized endoscope storage control.

In various embodiments, the controller may comprise one or more of a temperature sensor, a humidity sensor, an internal clock, and an RFID readout configured for reading out RFID equipped endoscopes and/or users, which can all be useful for documentation and process control.

In further embodiments, the controller may comprise a self-contained power supply, for example, a battery, or a connector to an external power supply.

The endoscope presence sensor may in embodiments be one of an optical sensor, a contact sensor, such as a switch, a weight sensor and a deformation sensor that can be located on or in vicinity to the receptacle. An optical sensor may be a photoelectric barrier to be blocked by a flexible endoscope being held by the receptacle or a photodiode or other photosensor that may be covered by an endoscope when held by the receptacle. A contact sensor may react to being in physical contact with an endoscope in the receptacle. While a weight sensor may be located to measure the weight of an endoscope held by the receptacle, a deformation sensor may be integrated into the receptacle to measure the deformation of the receptacle under the weight of an endoscope held by the receptacle. The types of sensors to be used for detecting the presence or absence of an endoscope is not limited to this list of sensors.

In embodiments, the flexible endoscope storage hook can comprise a fixation device, for example, prefabricated screw holes or a clamp, configured for aiding a fixing of the hook to a counterpart in or at a storage device or room.

Such object can also be solved by a flexible endoscope storage cabinet having one or more flexible endoscope storage hooks according to the previous disclosure, and by a flexible endoscope storage system having a central computer and one or more flexible endoscope storage hooks according to the previous disclosure, the central computer being configured to connect to the one or more flexible endoscope storage hooks, to receive data from the one or more flexible endoscope storage hooks and to centrally process the received data for documentation of storage of one or more flexible endoscopes.

### BRIEF DESCRIPTION OF THE DRAWINGS

Further features will become evident from the description of embodiments, together with the claims and the appended drawings. Embodiments can fulfill individual features or a combination of several features.

The embodiments described below, without restricting the general intent of the invention, based on exemplary embodiments, wherein reference is made expressly to the drawings with regard to the disclosure of all details that are not explained in greater detail in the text.

In the drawings:

FIG. 1 illustrates schematically an embodiment of a flexible endoscope storage hook,

FIG. 2 illustrates schematically an embodiment of a controller of a flexible endoscope storage hook,

FIG. 3 illustrates schematically an embodiment of a flexible endoscope storage cabinet, and

FIG. 4 illustrates schematically an embodiment of a flexible endoscope storage system.

In the drawings, the same or similar types of elements or respectively corresponding parts are provided with the same reference numbers in order to prevent the item from needing to be reintroduced.

### DETAILED DESCRIPTION

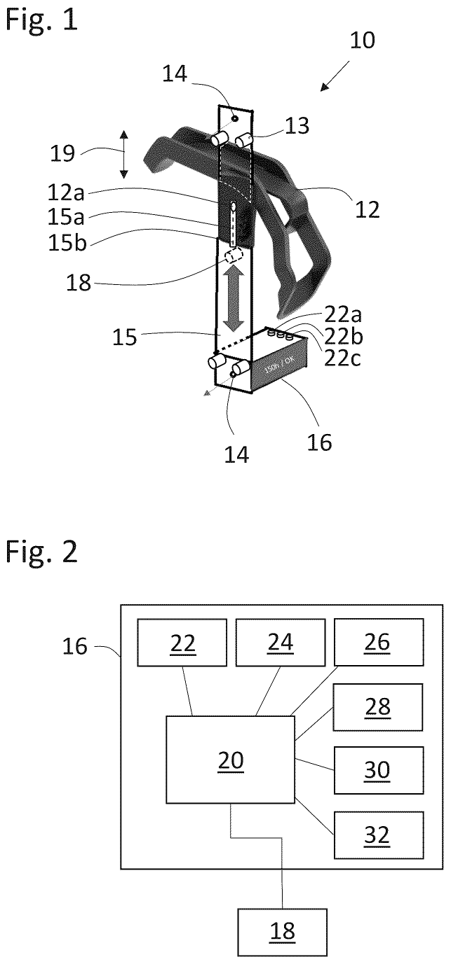

FIG. 1 illustrates schematically an embodiment of a flexible endoscope storage hook 10 having a receptacle 12 configured to accommodate a flexible endoscope (not shown) for the purpose of storing the endoscope until further use. The storage hook 10 has a back plate 15 with screw holes 14 for affixing the storage hook 10 to a wall of a cabinet or a storage room. Instead of screw holes 14, which may or may not have internal threading, the back plate 15 may alternatively be equipped with clamps configured to clamp onto matching holding structures in a cabinet. Furthermore, the storage hook 10 may have spacers 13 that allow the storage hook 10, such as its back plate 15, to be spaced at a distance from a cabinet wall. This spacing aids in keeping the storage hook 10 clean and disinfected, even if the cabinet wall may not be free of contamination.

For the purpose of illustration only, back plate 15 is only schematically shown, such as not to obscure features of the receptacle 12, a part of the receptacle 12 obscured by the back plate 15 is surrounded with dashed lines.

The receptacle 12 is slidably connected to the back plate 15 such as to slide down under the weight of a flexible endoscope, and back up after removal of the endoscope from the receptacle 12, which is illustrated by the double headed vertical arrow in FIG. 1. The sliding connection may be facilitated using a slot 15a in the back plate 15 or other guiding means, such as sliding rails. The receptacle 12 can be slidably held relative to the back plate 15 by one or more fasteners 12a slidably retained in the slot 15a. The one or more fasteners 12a, and thus, the receptacle 12, may be biased towards its uppermost position on the back plate 15 by a spring 15b or other elastic material. The one or more fasteners 12a or the receptacle may abut against a stop, such as a first end of the slot 15a, preventing the one or more fasteners 12a and receptacle from sliding up further under the biasing action of the spring. Likewise, there may be another stop, such as a second, opposite, end of the slot 15a, preventing the one or more fasteners 12a and receptacle 12 from sliding down past a lowermost position on the back plate 15, the one or two stops thus defining one or two well-defined end positions for the vertical movement of the receptacle 12.

A sensor 18 is configured to sense whether or not a flexible endoscope is present in or at the receptacle 12. For this purpose, the sensor 18 may be located at a position that is fixed relative to the back plate 15 and furthermore may be configured to react to the downward or upward movement of the receptacle 12 on the relative back plate 15 that indicates an endoscope being placed in or, respectively, removed from the receptacle 12. Such sensor 18 may be placed at one or both of the aforementioned vertical stop positions of the receptacle 12.

A non-limiting list of examples of a sensor 18 that can detect such movement of the receptacle 12 may include an electrical sensor such as an electrical limit switch or contact sensor positioned either such as to detect the receptacle 12 in its uppermost position, signaling an empty state of the receptacle 12, or positioned close to a lowermost position, signaling an occupied state of the receptacle 12, or optical sensors, such as a light barrier or a photoelectric cell, that may serve the same purpose, a magnetic sensor sensing the proximity of the receptacle 12 or a ferromagnetic part thereof, such as a strut or a screw that moves with the receptacle 12, a mechanical sensor, such as a push button or a lever being pushed by the receptacle 12 itself or a part mechanically connected to the receptacle 12 in either one of its extreme positions.

The sensor 18 may also be a weight sensor attached to the back plate 15 and supporting the weight of the receptacle 12. The weight sensor can detect the change in weight when a flexible endoscope is placed in the receptacle 12.

Alternatively, the receptacle 12 may be made of a deformable material which will deform somewhat under the weight of an endoscope. A deformation sensor, such as strain gauges, may detect such deformation 19 under weight, thus signaling the presence of an endoscope in the receptacle 12. In such a case, the receptacle 12 may be affixed to the back plate 15 without the ability of sliding up and down.

The storage hook 10 also comprises a controller 16 receiving the signal from sensor 18 and having components rendering the controller 16 capable of performing several functions that are built-in to commercially available drying and storage cabinets such as track and trace of individual endoscopes, controlling parameters such as storage time, operator identification or documentation of environmental parameters such as temperature and humidity. This, in essence, allows the user to perform operating a reprocessing workflow as it would be possible with commercially available drying and storage cabinets, thereby alleviating the substantial disadvantages self-built storage cabinets have with regard to documentation and process control and the negative impact this has on overall infection prevention and control.

As further illustrated in FIG. 1, the display may be a number of LEDs 22a, 22b, 22c, such as a green LED 22a, a blue LED 22b and a red LED 22c under the control of the controller 16. The green LED 22a may indicate that the receptacle 12 is empty and ready to receive a reprocessed flexible endoscope for storage. The blue LED 22b may indicate that a flexible endoscope is currently held in the receptacle 12 and ready to be used, whereas the red LED 22c may indicate that a flexible endoscope is currently held in the receptacle 12, but that a maximum storage time has been exceeded, the endoscope thus posing a contamination hazard, and the endoscope held in that receptacle needs to be reprocessed again before it is safe for use again.

FIG. 2 illustrates components of controller 16 that enable controller 16, and by extension storage hook 10, to carry out several of the above-described functions. A central unit of controller 16 is a processor comprising hardware, such as a CPU 20 that is connected to a number of peripheral units, several or all of which may be present in controller 16. The connections are indicated by lines connecting the CPU 20 with the peripheral units.

Controller 16 may have a display 22 configured to show information about an endoscope, storage related or other information to a user, either permanently or on request. Such a display 22 may be a touchscreen device, obviating the need for a separate manual input unit, which may alternatively be present in case the display 22 does not function as a touchscreen device.

Controller 16 may also comprise a wireless connection unit 24, for example a Wi-Fi or Bluetooth unit configured to connect controller 16 into a wireless local area network to which a central control unit, for example a computer, may be connected that performs a coordinating function across several similar flexible endoscope storage hooks 10.

Controller 16 may also be equipped with one or more of a temperature and/or timidity sensor 26, which may be a temperature sensor and a humidity sensor, internal clock 28, an RFID readout unit 30 and a power supply 32, which may be a self-contained power supply such as a battery or a connection to an external power supply. The RFID readout unit 30 may be configured to readout RFID tags of endoscopes for identification of individual endoscopes and/or RFID tags worn by users to document which user has used the storage hook 10 at what times and possibly with which endoscope.

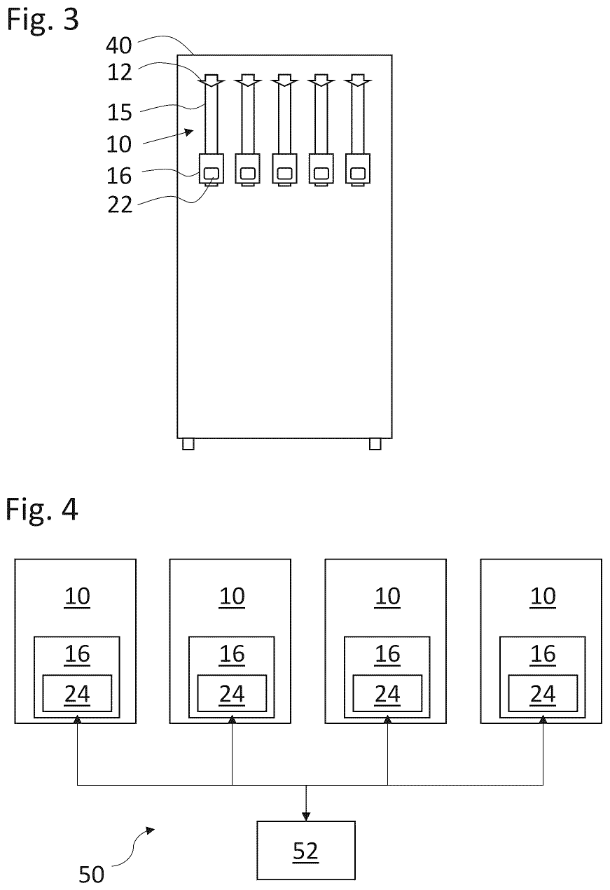

FIG. 3 illustrates schematically an embodiment of a flexible endoscope storage cabinet 40. The flexible endoscope storage cabinet 40 is in essence a cabinet with a multiple of flexible endoscope storage hooks 10 that may be configured according to FIG. 1 and FIG. 2 affixed to its back wall. The cabinet 40 itself has no controller of its own. Instead process control and documentation are provided by the flexible endoscope storage hooks 10 that are equipped with their own controllers 16, which have built-in process control and documentation functionality.

The receptacles 12 are not shown as such in FIG. 3, however, their downward movement under load by an endoscope is indicated with downward pointing arrows.

Furthermore, the controller 16 of each of the storage hooks 10 may have a display 22 configured to display information about the storage of the endoscope, such as storage duration since start of storage, remaining time to maximum storage time, type of endoscope or other appropriate information.

FIG. 4 illustrates schematically an embodiment of a flexible endoscope storage system 50 having a central computer 52 and several flexible endoscope storage hooks 10 according to the present disclosure, for example such as are shown in FIGS. 1 and 2 and used in FIG. 3. Data connectivity between the hooks 10 and the central computer 52 may be provided by wireless connectivity units 24 contained in the respective controllers 16 of the respective flexible endoscope storage hooks 10 of system 50. The central computer 52 may be configured to provide a centralized documentation and process control needed in the context of clinical endoscopy with multiple endoscopes.

Alternatively, a flexible endoscope storage system may be configured without a central computer 52. In that case, the several flexible endoscope storage hooks 10 may interconnect with each other via their wireless connection units 24 and provide either a distributed process control and documentation or, in the context of establishing a connection, broker a specific one of the flexible endoscope storage hooks 10 to act as a central process control and documentation unit receiving and processing data from all the other connected flexible endoscope storage hooks 10, thus providing the same functionality as the central computer 52 of the embodiment of FIG. 4.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### List of Reference Signs

10 flexible endoscope storage hook
12 receptacle
12a fastener
13 spacer
14 screw hole
15 back plate
15a slot
15b spring
16 controller
18 sensor
19 deformation
20 CPU
22 display
22a, 22b, 22c LEDs
24 wireless connection unit
26 temperature sensor
28 internal clock
30 RFID readout unit
32 power supply
40 storage cabinet
50 storage system
52 computer

What is claimed is:

1. A storage hook for storing a flexible endoscope, the storage hook comprising:
a receptacle for storing the flexible endoscope,
an endoscope presence sensor configured to detect a placement or removal of the flexible endoscope in the receptacle, and
a controller including a processor comprising hardware, the processor being configured to receive an input signal from the sensor and to output an output signal based on the input signal from the sensor.

2. The storage hook of claim 1, wherein the output signal of the processor is for one or more of process control and documentation of the flexible endoscope.

3. The storage hook of claim 1, wherein the controller further comprises a display and a wireless connection unit.

4. The storage hook of claim 1, wherein the controller further comprises at least one of a temperature sensor and a humidity sensor.

5. The storage hook of claim 1, wherein the controller further comprises an internal clock.

6. The storage hook of claim 1, wherein the controller further comprises an RFID readout configured to read information output from the flexible endoscope.

7. The storage hook of claim 1, wherein the controller further comprises a self-contained power supply.

8. The storage hook of claim 7, wherein the self-contained power supply comprises a battery.

9. The storage hook of claim 1, wherein the controller further comprises a connector configured to be connected to an external power supply.

10. The storage hook of claim 1, wherein the endoscope presence sensor is one of an optical sensor, an electrical or mechanical contact sensor, a weight sensor, a magnetic sensor and a deformation sensor operatively connected to the receptacle.

11. The storage hook of claim 10, wherein the contact sensor is a switch.

12. The storage hook of claim 1, further comprising a back plate for supporting the receptacle.

13. The storage hook of claim 12, wherein the receptacle is slidably connected to the back plate, and contains a spring biasing the receptacle against the back plate into an uppermost position.

14. The storage hook of claim 12, wherein the back plate having one or more screw holes configured to fix the back plate to one of a storage device or room.

15. The storage hook of claim 1, further comprising one or more spacers configured and positioned on the storage hook to provide a spacing between the hook and a cabinet wall.

16. A storage cabinet comprising one or more storage hooks according to claim 1.

17. A storage system comprising:
a central computer; and
one or more storage hooks according to claim 1,
wherein the central computer being connected to the processor of the one or more storage hooks, to receive data from the one or more storage hooks and to centrally process the received data for documentation of storage of one or more corresponding flexible endoscopes.
